(19) United States
(12) Patent Application Publication
(10) Pub. No.: US 2011/0283785 A1
(43) Pub. Date: Nov. 24, 2011

(54) Testing Method and Kit for Detecting Lead, Mercury, and Chromate in Paint , Varnish, and Other surface Coatings

(76) Inventors: Daniel P. Askin, Milwaukee, WI (US); Zsuzsanna Dorko, Milwaukee, WI (US)

(21) Appl. No.: 13/111,722

(22) Filed: May 19, 2011

Related U.S. Application Data
(60) Provisional application No. 61/346,291, filed on May 19, 2010.

(51) Int. Cl. G01N 33/00

(52) U.S. Cl. 73/150 R

(57) ABSTRACT

A testing kit and method of testing for the presence of lead mercury and chromate in surface coatings is provided that can accurately determine the presence of lead, mercury and chromate in the surface coating without interference from other constituents of the surface coating. The kit includes an alkaline caustic that is utilized to dissolve the coating material to enable any lead or mercury present in the coating material to react with a subsequently added second solution containing sulfide ions, while preventing interference of other ionic species with the interaction of the lead and sulfide. The sulfide ions can then react with the lead or mercury in order to produce a dark color for the resulting solution. The resulting color can then be compared against a color standard to determine the amount of lead or mercury present in the sample. The kit and method also enables a determination for the presence of chromate by dissolving the coating material in the alkaline solution and observing the development of a characteristic pale yellow color.

### CROSS-REFERENCE TO RELATED APPLICATIONS

This application claims priority from U.S. Provisional Application Ser. No. 61/346,291, filed on May 19, 2010, the entirety of which is hereby expressly incorporated by reference herein.

### FIELD OF THE INVENTION

The present invention relates to testing methods, and more specifically to a testing method and kit for determining the presence of lead and chromate in coatings, such as paint and varnishes.

### BACKGROUND OF THE INVENTION

Metal compounds have been used in paints and coatings for as long as paints and coatings have been produced. These metal compounds provide coloration along with many other desirable properties, including but not limited to the ability to hide an underlying surface color; as well as provide a coating in a color of choice and to protect the coated material from corrosion or premature failure from environmental causes. This simple test provides a rapid means for the determination of the presence of the lead in coatings, and also to provide some additional information on the concentration of lead in the coating, whether or not the lead form(s) present includes lead chromate while excluding all of the other substances ever used in paint from interfering with the test.

Lead has been used for hundreds of years in coatings, as a pigment in paints, as a corrosion inhibitor and as a drier in paints and varnishes. The primary reasons for its use were for its novel properties including the resulting durability of the finished coating, the broad spectrum of colors that could be made using lead, and its corrosion, water and weather resistance. When the coatings formed with lead therein age, they deteriorate and consequently create lead-containing or lead-contaminated dust. When these coatings are disturbed by cutting, drilling, sanding, or other methods commonly employed to remove building materials; the lead dust is released from the coating as a result of its disintegration and can readily disseminate and contaminate large areas. This has become problematic, because lead-contaminated dust has been identified as a significant health hazard, particularly to children.

Over the years, in order to achieve the benefits of lead in various types of coatings, different forms of lead and lead-containing compounds have been incorporated into the coatings. The range of lead compounds that have been used in coatings includes, but is not limited to:
Lead Sulfate
Lead Chromate
Lead Monoxide (Litharge)
Lead Dioxide (Brown)
Red Lead Oxide
Lead Silicate
Lead Sulfate blue basic—Mixture
Lead Linoleate and Lead Naphthenate
Lead Carbonate

While each type of lead compound may have certain properties that differentiate it from the remainder of the compounds, the commonality of the presence of lead in each compound renders each of these compounds a health risk.

Chromates have been used in paints and still are in use today, similar to lead. Historical uses included lead chromate and zinc chromate as both a pigment and corrosion inhibitor. Zinc chromate is still in common use today in metal primers and lead chromate is still sometimes used in primers for structural steel. Sodium Chromate and Sodium Dichromate have been used and may still be in use as colorants in paint. All chromates contain Chromium in the +6 valence state (commonly known as Chrome VI, Hexavalent Chromium or Hex Chrome). OSHA regulates exposure to Hexavalent Chromium due to its strong carcinogenicity. Upon deterioration of the coating over time, Hex Chrome contaminated dust is generated as well as during removal of the coating or removal of painted components.

OSHA regulates exposure to hexavalent chromium in general industry (29 CFR 1910.1026), shipyards (29 CFR 1915.1026), and construction (29 CFR 1926.1126). The occupational Permissible Exposure Limit (PEL) for Chrome VI is 2.5 micrograms per cubic meter and the occupational PEL for lead is 5.0 micrograms per cubic meter.

Mercury compounds were used in paints as a pigment and they were also used as a bactericide and mildewcide in latex paints until they were banned in 1991

To address the issue of the health risks from lead-containing dust from these coatings, the United States Environmental Protection Agency (USEPA) has recently published regulations governing the appropriate methods and procedures to be used during remodeling and renovation construction work for residential housing. These regulations stipulate that for all housing built before 1978 the surfaces to be disturbed during renovation or remodeling activities must be checked for the presence of lead in the surface coatings. There are several ways this can be accomplished.

For example, detection of lead in cured surface coatings can be done by a certified sampling technician collecting a paint sample in accordance with the ASTM Standard Practice for Field Sampling of Coating Films for Analysis for Heavy Metals (Designation D 5702-07), and having the paint sample analyzed by Atomic Absorption Spectroscopy (AAS) or other appropriate laboratory method. While this method can be very accurate, it is heavily dependent on the skill used to collect the sample in order to obtain a representative sample of the surface coating. Additionally, the testing procedure is expensive, with a typical lab analysis for lead in the surface coating specimen costing between $15 and $35 per sample. Yet another disadvantage of this method is the time delay between the time the sample is collected and the results are received from the lab, which can be days or weeks depending upon the number of tests to be run. This can significantly delay the project, resulting in highly increased costs.

More recently, and as an alternative to (AAS), X-Ray Fluorescence (XRF) Spectrometers have been adapted for the detection of lead in surface coatings. Nevertheless, while this method gives instant results, i.e., within minutes, there are some significant drawbacks with this method as well. In particular, the spectrometer instrument is expensive, and requires a trained operator who also possesses a nuclear license from the Nuclear Regulatory Commission.

The least expensive alternative method is to use a chemical spot test which provides fast results. However, the various chemical spot tests available are either unable to consistently detect lead chromate, which has historically been utilized as a common pigment, or difficult to detect lead-containing driers, such as lead linoleate and lead naphthenate. Further, while they are marketed as providing fast results, when used to determine the presence of lead chromate, the currently commercially available chemical spot test kits either do not detect lead chromate, or if they do, the test kits can take anywhere from ½-24 hours to have a conclusive test.

A further limitation of the existing chemical spot tests includes interferences from other common components of paint and varnish coatings and the substrates to which the coatings have been applied.

In addition, and perhaps most importantly, the chemical spot tests have various issues regarding their detection limits. For example, the detection limits for the tests can vary with the form of lead that is present, rendering the tests unusable for certain lead-containing compounds, and have been found to be insufficiently accurate (i.e., none of the chemical test kits were able to achieve low (5% or lower) rates of both false positive and false negative results) by: 1) the U.S. Consumer Product Safety Commission (CPSC) as reported in: CPSC STAFF REPORT: EVALUATION OF LEAD TEST KITS, October 2007; 2) the National Institute for Standards and Technology and the U.S. Department of Housing and Urban Development Office of Lead Hazard Control as reported in: NISTIR 6398, “Spot Test Kits For Detecting Lead in Household Paint: A Laboratory Evaluation,” NIST, May, 2000; 3) “The Use of Manufactured Samples for Evaluating Spot Test Kits for Detecting Lead in Household Paints,” HUD Technical Report, U.S. Department of Housing and Urban Development, Washington, D.C., December 2000; and 4) “Lead-based paint testing technologies: summary of an EPA/HUD field study,” American Industrial Hygiene Association Journal, Vol. 60, No. 4 (July/August 1999), pp. 444-451, each of which are expressly incorporated by reference herein in their entirety.

Additionally, the chemical spot tests do not allow for determination of lead at the thresholds defined in US and state regulations. Specifically, the Department of Housing and Urban Development (HUD), defines lead-based paint as paint or other surface coatings that contain lead equal to or exceeding 1.0 milligram per square centimeter (mg/cm²) or 0.5 percent by weight (equivalent to 5,000 parts per million or ppm). The (CPSC) definition of lead containing paint means paint or similar surface coating materials in which the lead content exceeds 0.06 percent by weight (16 C.F.R. Part 1303) (equivalent to 600 ppm). The States of Maryland and Wisconsin as well as several municipalities define lead based paint at the level of 0.7 mg/cm². (See also: NISTIR 6398, supra).

The two types of chemical spot test kits for the detection of lead compounds in paint currently available are based on chemical reactions of either the rhodizonate ion, which produces a pink or red color in the presence of lead, or the sulfide ion, which produces a gray, brown or black color in the presence of lead.

Most test kits also come with extensive instructions for use and interpretation of results, as referenced in the CPSC Evaluation mentioned previously. Further, the existing technology for chemical spot tests is summarized in the US Army Paint Manual:
“(1) Sodium sulfide. Spot testing using sodium sulfide is a qualitative method for determining the presence of lead. One method of conducting the test involves cutting a beveled scribe through the coating down to the substrate, exposing each of the layers within the coating system. A 6 to 8 percent aqueous solution of sodium sulfide is deposited across the scribe, and a reaction occurs between lead and the sulfide ion to form black lead sulfide. The change to a gray/black color typically occurs within seconds. If the existing coating is white, or a light color, and only one or two layers, this test may provide a viable means for determining whether or not lead is present. However, industrial paints typically are many layers, only a few of which may contain lead. An adequate area of each layer must be exposed to make the visual determination of color change. Additionally, the interpretation of a color change may be difficult with darker coatings, particularly when only thin layers are exposed for testing. The tester also must be able to distinguish between the darkening of a layer of the coating that may occur from the wetting solution compared to a darkening caused by exposure to sodium sulfide.
(2) Rhodizonate. Another spot test relies on the reaction between lead and the rhodizonate ion to precipitate a pink complex. The coating film is cut or sanded away to the substrate to expose a cross-section of the film, and a solution of rhodizonate is directly applied using a special applicator or applied to a filter paper that is placed against the surface. The reaction, which may occur instantly or require a few minutes, creates a rose-red coloration that indicates the presence of lead.” (EM 1110-2-3400, 30 Apr. 1995 US Army Corps of Engineers
Engineering and Design Painting: New Construction and Maintenance)

The USEPA has published a regulation at 40 CFR §745.88 detailing their criteria for acceptable test kits for lead in coatings:

(c) Response Criteria:
(1) Negative response criteria. For paint containing lead at or above the regulated level, 1.0 mg Pb/cm² or 0.5% by weight, a demonstrated probability (with 95% confidence) of a negative response less than or equal to 5% of the time.
(2) Positive response criteria. For paint containing lead below the regulated level, 1.0 mg Pb/cm² or 0.5% by weight, a demonstrated probability (with 95% confidence) of a positive response less than or equal to 10% of the time.

Both types of tests are limited by interfering substances that can cause inaccurate results. In particular, with sulfide ion kits, the presence of certain ions, for example, iron, will give a false positive result. As a result, sulfide test kits pose problems in that they form dark complexes with many other inorganic components of paint and other surface coatings as shown in Table 1:

TABLE 1
Elements Forming Complexes with Sulfide

| ELEMENT | COLOR OF COMPLEX |
|---|---|
| Antimony | Black, Red |
| Bismuth | Black, Brown, Gray |
| Cadmium | Black |
| Chromium | Black, Brown, Gray |
| Cobalt | Black, Gray, Red |
| Copper | Black |
| Iron | Black, Green, Yellow |
| Lead | Black |
| Manganese | Black, Green, Pink |
| Mercury | Black, Red |
| Molybdenum | Black, Brown, Gray |
| Nickel | Black, Gray, Yellow |

Thus, each of these paint and surface coating components, if present, will provide a false positive with regard to the detection of any lead present in the surface coating utilizing any of the sulfide testing methods known in the previous art. Another limitation of the previous sulfide methods is that they were limited in their use on paints of a dark black, grey or brown coloration.

Additionally, kits incorporating the rhodizonate ion in the testing reaction can give a false negative result when the only lead present is in the form of lead chromate, or a false positive result in the presence of barium. As stated in the CPSC Evaluation, rhodizonate test kits pose problems in regards to selectivity, as the rhodizonate is known to react with sulfate as found in plaster and wall board. This reaction depletes the amount of rhodizonate available to react with the lead and thus can cause false negative results. The National Institute of Occupational Safety and Health (NIOSH) Manual of Analytical Methods, Fourth Edition, 1996, Methods 7700 and 9105, lists Cd²⁺ and Sn²⁺ as interferences. The EM Quant Lead Test Catalog No. 10077 from Gallade Chemical Incorporated, Santa Ana, Calif., lists the anions iodite, oxalate, sulfide, and sulfite as significant interferences, and the cations Cu²⁺, Sr²⁺, Fe³⁺, and Ba²⁺ as significant interferences. Of these potential interferents, cadmium, iron, and barium are likely to be found in old paints and therefore be a potential problem. Another known problem with all of the rhodizonate tests is the frequency of red, pink or orange pigments. These pigments can often leach into the test reagents and generate a pink or red color indicating a potentially false positive result. Yet a further problem with the rhodizonate tests is the prevalence of color blind individuals who are unable to see the difference between a positive and negative test.

One improvement to these chemical spot tests is found in Cole U.S. Pat. No. 6,489,170 which claims to have overcome the limitation of interfering ions in the traditional rhodizonate tests by substituting a stronger acid, such as hydrochloric (HCl) acid to avoid these interferences, and in particular barium. However, the test disclosed in the Cole patent does not provide a scale of lead concentration that is found in the surface coating sample, but only a basic positive or negative result, nor does it resolve interferences from red, blue or purple paint pigments or coloration, nor does it address the detection of all of the crystalline forms of lead chromate.

A common difficulty with all of the previous chemical spot tests to detect lead in paint has been to obtain a reaction that produces a consistent color change reaction regardless of the form of lead present, the color of the paint and regardless of the substrate in a simple, yet rapid test.

Accordingly, a need exists for a relatively simple testing kit and method of detecting lead in a dried paint film or other surface coating that is accurate, easy to administer, safe, and inexpensive. Preferably, this kit and method should be capable of detecting low quantities of lead in a coating, to qualitatively and at least semi-quantitatively identify the presence of lead without interference from other elements or compounds, such as barium or iron, without interference from the paint color, and to meet the need for a test which can provide information on the amount of lead present in a coating. Additionally, the kit and test should be able to be adjusted to determine lead levels at specific, predetermined concentration thresholds over the entire range of 0.1 mg Pb/cm² or to 3.0 mg Pb/cm² by manipulation of test kit variables, or over a wider range if all of the test kit variables are adjusted.

### SUMMARY OF THE INVENTION

According to one aspect of the present disclosure, a testing kit and method of testing for the presence of lead or mercury in surface coatings is provided that can accurately and precisely determine the presence of lead in the surface coating without interference from other constituents of the surface coating or its color. The kit includes an alkaline solution that is utilized to dissolve the coating material to enable any lead or mercury present in the coating material to react with a subsequently added second solution containing sulfide ions, while preventing interference of other ionic species with the interaction of the lead or mercury and the sulfide ions. The sulfide ions can then react with the lead or mercury in order to produce a dark color in the resulting solution. The resulting color can then be compared against a color standard to determine the amount of lead present in the sample.

According to another aspect of the present disclosure, the resulting solution can be determined to have remained completely colorless which indicates that no lead was present in the paint sample, or at least below the detection limit of the method.

According to yet another aspect of the present disclosure, the kit and testing method can result in the detection of both lead and sodium chromate (CrO₄⁻²) and dichromate (Cr₂O₇⁻²) in coatings by the appearance of a faint yellow color of the solution when it is mixed with the alkaline solution before the addition of the sulfide ion. These chromates and dichromates are soluble in alkaline solution and insoluble in acidic solution. Once dissolved in an alkaline solution containing sodium ions, they create a solution of Sodium Chromate (Na₂CrO₄). Sodium chromate is a yellow dye and forms a distinctly clear yellow solution.

According to yet another aspect of the disclosure is the method of leaching the metal of interest from a paint or other coating sample in a suitable extraction solution and reacting the leachate with any of the chemicals that can form a colored compound or complex with the metal of interest in vial 18. The extraction solution may be an acidic solution, but preferably is an alkaline aqueous solution, or a two phase solution including an organic or non-polar solvent in combination with an aqueous extracting solution. Following extraction, the extract is reacted with a reagent that forms a colored solution in a clear vial that produces a continuously variable color intensity that correlates with the concentration of the metal of interest.

According to yet another aspect of the present disclosure the presence or absence of lead or mercury in a dried coating film can be determined by:
1. Cutting a ¼″ long notch through all the layers of a dried coating film so as to expose all of the layers of the coating.
2. Applying the alkaline extracting solution to the exposed edges of the coating film.
3. Either before or after the extracting solution dries, applying a drop of aqueous sulfide to the exposed edges of the coating film.
4. Observing the presence or absence of the characteristic brown to black color that indicates the presence of lead and or mercury.

According to still a further aspect of the present disclosure, the method for detecting the presence of lead or mercury involves placing the solution including reagent(s) and the sample of interest in a single, clear and transparent vial, and visually evaluating the presence of lead or mercury in the sample by light transmission and/or reflectance through the solution in comparison with a color test standard.

Numerous other aspects, advantages and features of the present disclosure will become apparent in the following detailed description taken together with the drawing figures.

### BRIEF DESCRIPTION OF THE DRAWINGS

The drawings illustrate the best mode currently contemplated of practicing the present disclosure.

In the drawings:

FIG. 1 is an isometric view of a test kit assembled according to the present invention;

FIG. 2 is top plan view of a sample removal device and push rod included in the kit of FIG. 1;

FIG. 3 is an isometric view of a the device of FIG. 2 removing a sample from a vertically oriented sample area;

FIG. 4 is a cross-sectional view along line 4-4 of FIG. 3

FIG. 5 is a cross-sectional view of the push rod displacing the sample from within the sample retrieval device of FIG. 2;

FIG. 6 is a top plan view of the removal of a sample from a horizontally oriented sample area;

FIG. 7 is an isometric view of a sample being introduced into a test bottle containing a test solution from the collection paper;

FIG. 8 is a front plan view of a reagent in a reagent bottle being added to the test bottle including the sample; and

FIG. 9 is a front plan view of the evaluation of the final solution in the test bottle with a test standard on the test bottle.

### DETAILED DESCRIPTION OF THE INVENTION

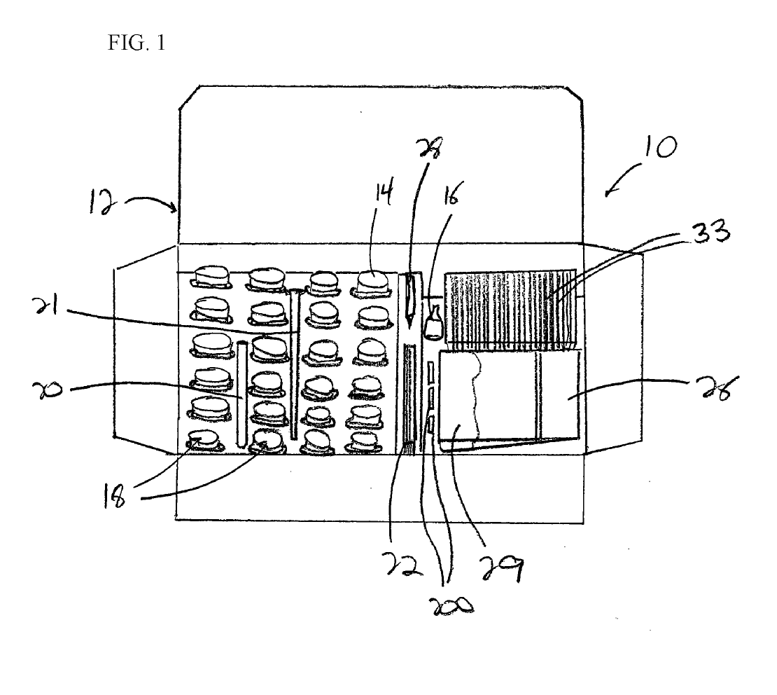

With reference now to the drawing figures in which like reference numerals designate like parts throughout the disclosure; a kit formed according to the present invention is shown generally at 10 in FIG. 1. The kit 10 includes a reclosable container 12 within which are disposed a pair of bottles 14 and 16 containing the reactant solutions for reacting with the lead, one or more clear and colorless reclosable sample test vials 18 within which a sample of the surface covering is placed to be contacted with the reactants, a sample retrieval or removal device 20, a push rod 21 and a number of pieces of sample collection paper 22 for retrieving the sample 23 from the device 20, and a color test standard 24 (FIGS. 7-9).

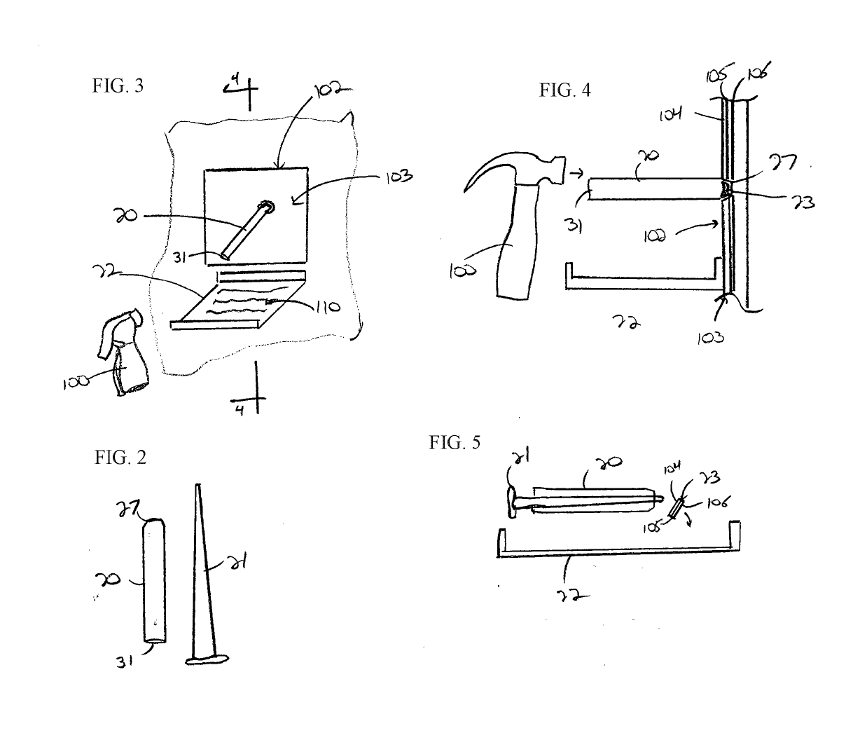

Prior to obtaining the sample 23 of the surface coating, the sample area 102 and the devices 20 and 21, best shown in FIG. 2, are cleaned to remove any surface dust or other contaminants, as this can include lead dust from other sources than the current paint or other coating on the surface to be sampled. This is most conveniently done with a pre-moistened wipe towelette 33 which may also be provided in the kit 10, which in one embodiment can be a D-Wipe® towelette, sold by ESCA Tech, Inc. of Milwaukee, Wis.

Concerning the process for or step of obtaining the sample 23 of the surface coating, it is important to ensure that a true cross-section of the coating is obtained, such that all paint layers 104-106 of the coating 103 on the sample area 102 are present in the sample 23 and exposed so they may interact with the test solutions in vial 18. While there are numerous devices and methods available that can be employed to collect a sample 23 through all of the paint or other coating layers 104-106, one embodiment of the kit 10 includes a sample retrieval device 20 formed as a hollow metal tube composed of brass, aluminum, stainless steel or other metal alloy. A sample cutting edge 27 of the device 20 is inwardly tapered to an angle of between about twenty (20) and sixty (60) degrees, and preferably about twenty (20) to thirty-five (35) degrees in order to effect a clean cut into the surface coating by the device 20. The reproducibility of the sample size obtained by the device 20 is essential to determining whether the coating contains lead above or below the specified limit. This simple to use device 20 removes most of the variability inherent in existing methods of paint sample collection and provides a consistent size sample through as many as 10 layers of paint and exposes a uniform surface area of each paint layer to the reactants in the test. If more than 10 layers are present, then the sample can be acquired in sequential sample acquisition steps until all of the paint is removed by the sampling device 20. By selection of different, reproducible sample sizes, the threshold between a positive and negative result over the range of 0.1 mg/cm² and 3.0 mg/cm² can be determined. For a detection limit of 1.0 mg/cm² to meet the USEPA criteria for acceptable test kit standards, the inside diameter of the device 20 should be about 0.186″, with a wall thickness of between about 0.03″ to 0.06″ when used with the other test kit component sizes, concentrations, quantities and compositions listed here. This sample diameter of 0.186″ is the minimum size sample that can be conveniently handled and manipulated that will still provide a sufficiently low detection limit.

As shown in FIGS. 3 and 4, when the edge 27 of the device 20 is placed against the coating, and the opposed end 31 is tapped with a hammer 100 or other suitable instrument, a well-defined sample 23 is formed by and lodges in the interior of the device 20. This ability of the device 20 to form the well-defined sample 23 is important to enable reproducible samples to be obtained from the surface coating 103 on the sample area 102, and to remove portions of all of the layers 104-106 of the coating 103 cleanly from the substrate. For those instances where some of the coating layer remains on the coating surface, a suitable implement such as a single edge razor blade with handle or a box cutter 28, optionally included in the kit 10, can be used to recover any of the paint or coating layers that are not contained within the sample retrieval device 20.

In addition, when performing the step of obtaining the sample 23, to ensure that all of the sample 23 is recovered, a small piece of paper, such as a sheet of sample collection paper 22, optionally with a light adhesive along one side, is folded and positioned on the surface adjacent to the location of the sample 23 in the sample area 102 to catch any portion of the sample 23 not captured within the device 20, and can then be used to collect all of the sample 23 for efficient transfer into the sample test vial 18. The sample collection paper 22 is preferably made of standard paper or glassine paper and can have various types of indicia 110 printed thereon, including, but limited to, instructions for using the paper 22, obtaining the sample 23 and performing the test procedure. If the sample area is vertically oriented, the adhesive on the paper 22 is utilized to adhere the paper 22 directly below the sampling area to collect any fugitive portion of the sample 23 when the paper 22 is adhered to the area 102 and folded into a tray-like configuration, as shown in FIGS. 3-7. Alternatively, the paper 22 that does not include an adhesive can be adhered to the surface with adhesive tape or other suitable means. If the sample area 102 is horizontally oriented, it is useful to have the paper 22 positioned adjacent to the sample area 102 to collect the sample 23 in the same manner one would use a dust pan. If the sample area 102 is overhead, a small piece of tape may be placed over the open end 31 of the retrieval device to contain any sample that would fall through the device 20. In one embodiment the tape can be painter's tape, or masking tape or apiece of the adhesive edge of the paper 22 with dimensions of approximately ¼″×¼″.

In order to remove the sample 23 from the device 20, any suitable instrument can be used, or the device 20 can simply be shaken in order to dislodge the sample 23 from within the device 20. One preferred method for removal of the sample 23 from the device 20 is with a push rod 21, as shown in FIG. 5. This push rod 21 is formed to have an outer diameter less than that of the interior diameter of the device 20 and a length greater than that of the device 20 such that the rod 21 can be positioned within the device 20 and manipulated to push the sample 23 out of the device 20 onto a sheet of the collection paper 22. The rod 21 is preferably of a non-porous, smooth material, such as molded plastic, to make it easy to clean between samples. When tape is used to contain a sample as in sampling an overhead surface, the push rod 21 can be used to transfer the tape along with any portion of the sample to the collection paper 22. The presence of the small piece of tape will not interfere with any aspect of the subsequent test.

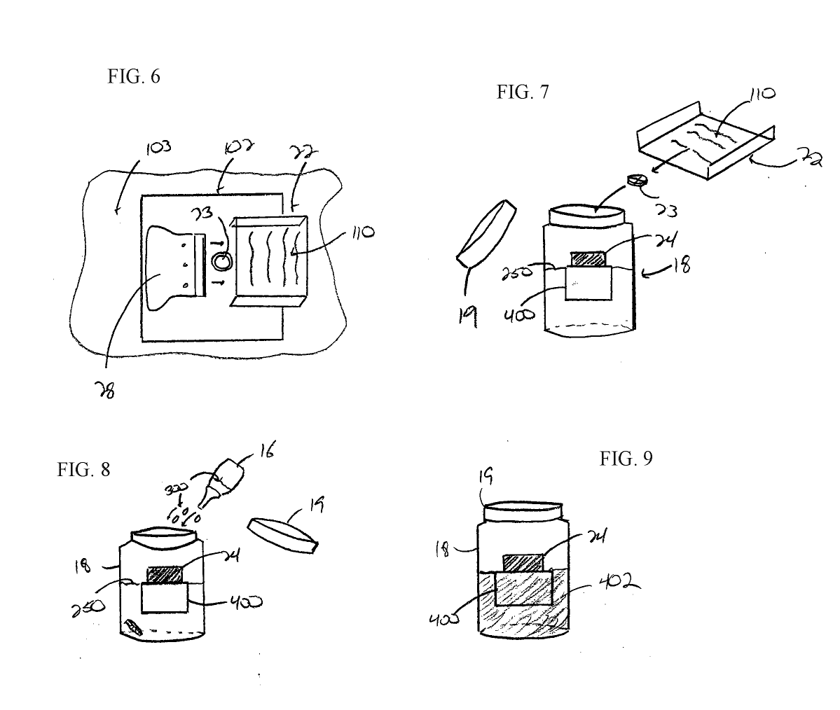

If the sample 23 is not cleanly and completely removed from the sample area 102, such that some or all of the sample 23 remains in the sample area 102, the razor 28 can be used to score, cut and/or scrape the sample 23 and all of the layers 104-106 away from the sample area 102 onto the collection paper 22, as shown in FIG. 6.

After the sample 23 is removed from the device 20 onto the sheet 22, and after optionally cutting the sample 23 into smaller pieces, e.g., quartering the sample 23, the sample 23 can then be placed into the sample test vial 18 (FIG. 7). Prior to the introduction of the sample 23 into the vial 18, a predetermined amount of a first solution 250 from bottle 14 is introduced into the vial 18. Alternatively, the vial 18 can be precharged with the necessary amount of first solution 250, approximately 12 ml for the size of the sample 23 in the illustrated embodiment, eliminating the need for the bottle 14 in the kit 10 and assisting in having a precise amount of the first solution 250 available for use in the lead test. Further, in this embodiment the kit 10 can be formed with multiple vials 18 with removable caps 19, each having the required amount of the first solution 250 contained therein.

The first solution 250 in bottle 14 is an alkaline or caustic solvent solution that is used to deteriorate the paint sample and selectively dissolve the lead present in the sample 23 in order to enable the lead to react with the second solution 300. While a number of different alkaline solutions can be utilized for this purpose, in one embodiment the first solution 250 is formed as a colorless, aqueous solution of between 0.15% w/w and 15% w/w of an inorganic caustic and, optionally, between 0.15% w/w and 15% w/w of an alkanolamine. In another embodiment, the solvent is a mixture of an alkali metal hydroxide, such as sodium hydroxide or potassium hydroxide, among others, and an alkanolamine, such as diethanolamine or triethanolamine, among others. The preferred alkali metal hydroxide is sodium hydroxide at a concentration of 1% w/w. While this embodiment of the first solution 250 is effective by itself, the test performance is improved by the addition of 3% w/w triethanolamine (TEA) to the first solution, with the percentages of the components optimized for a lead determination threshold of 1.0 mg Pb/cm² which is the standard mandated by the USEPA for an acceptable testing kit.

The use of an alkaline solvent provides a means to selectively leach any lead or mercury out of the coating sample 23 because the other metals that could be present and interfere with the sulfide reaction are either insoluble or are only very sparingly soluble in alkaline solution, thereby greatly reducing the interference of these metals with the test results. The addition of the TEA has several other benefits, including speeding up the leaching time by providing reserve alkalinity, inhibiting the suspension of other pigments into the test solution 250, partially dissolving any organic component of the surface coating or paint matrix, including any organic lead or mercury compounds present thereby allowing the lead to or mercury be released into the solution 250 more quickly, and further limiting the solubility of the interfering metals. Of all the metals that have historically been used in coatings, including paints and varnishes, the metals that form dark colored complexes with sulfide ions, some of which are identified above in Table 1, are excluded from reaction with the sulfide ion by the use of the dilute mixture of alkali metal hydroxide and an alkanolamine, with the exception of lead and mercury. However a false positive test result for a coating that is lead free but contains mercury is not considered a limitation of the test, due to the health hazards presented by mercury. If the coating sample 23 tested contains mercury, which was also commonly used in other coatings, such as marine coatings, and results in a positive test for lead, it would be equally or more important to use lead safe work practices when this coating is disturbed. Thus, throughout this disclosure, the application of the test kit 10 for determining the presence of lead also can additionally be interpreted to cover the use of the test kit 10 for determining the presence of mercury.

The benefit to this caustic extracting solution is its ability to dissolve all of the forms of lead and mercury, including lead chromate and lead dichromates which are soluble in alkaline solution. The chromate then combines with the free sodium in the extracting solution giving the distinct clear, yellow dye coloration of the sodium chromate.

Another benefit and an important functionality of the alkanolamine is the ability to liberate the organic lead driers from the dried paint matrix of which lead napthenate and lead linoleate were the most common for subsequent reaction with the sulfide.

After the sample 23 is introduced into the test vial 18 containing the first solution 250, the vial 18 is closed using the cap 19 and shaken for approximately 10-15 seconds to enable the first solution 250 to effectively leach the sample 23. For most samples it takes no more than 10-15 seconds to extract 90% of the available lead and chromate from the sample 23. After shaking the sample 23 in the vial 18, if the solution turns a pale yellow, this indicates that chromate is present in the sample 23, such that if the sample 23 is subsequently also positive for lead, it is known that the lead is present in the form of lead chromate. Since chromates are composed of hexavalent chromium and oxygen, any subsequent paint removal or disruption of a chromate coating regardless of whether it also contains lead will liberate carcinogenic hexavalent chromium dust.

Subsequently, as shown in FIG. 8, the cap 19 is removed and approximately five (5) to six (6) drops, each drop having an approximate volume of 0.1 mL, of the second solution 300 from the bottle 16 is added to the test vial 18 resulting in an addition to the first solution 250 in vial 18 of 0.01 mg of sulfide ion, which the minimum amount required for the detection of lead in relation to the selected size of the sample 23, the volume of first solution 250 in vial 18, the size of the vial 18 and the color standard 24. The second solution 300 is a clear, colorless aqueous solution of a water soluble sulfide compound, such as sodium sulfide, ammonium sulfide, carbon disulfide or dimethyl sulfide, capable of providing a sulfide ion at a concentration between about 0.01% w/w and 15% w/w in the second solution. In another embodiment, the second solution includes about 0.03% w/w to about 5.0% w/w of the sulfide ion, with the second solution tailored to the USEPA detection standards including about 0.5% w/w of ammonium sulfide, which is preferred as the sulfide component as it is easier to make a standard concentration in solution and is more stable. Further, in additional embodiments, the second solution 300 can be formed with any concentration of sulfide ion in the second solution greater than 450 ppm in order provide the required 0.01 mg of sulfide ion in vial 18 The sulfide concentration in the second solution 300 can potentially be less than this if additional drops of the second solution 300 are added to the first solution 250 containing the sample 23 to reach the required 0.01 mg of sulfide ion in vial 18. Changes to one or more of the size of the sample 23, the volumes of the first and second solutions 250, 300, the size of the vial 18 and/or the color standard 24 will result in corresponding changes to the other parameters for the test.

After adding the second solution 300, the vial 18 is closed using the cap 19 and shaken for approximately 10 seconds. As illustrated in FIG. 9, if lead is present, the combined solution 402 will immediately darken and the color of the resulting solution 402 is then compared to the color test standard 24 to determine the lead concentration detected. The vial 18 is one inch in diameter, and can be formed of glass or plastic, as long as it is colorless and optically clear so that it will not interfere with comparing the color standard 24 to the solution in the vial 18, and that the vial 18 has a uniform wall thickness for uniform light transmission through the walls of the vial 18.

Further, in one embodiment of the vial 18, the color standard 24 is printed on or applied to the vial 18 directly above the viewing area or window 400 on the vial 18 so during evaluation of the result both the color standard 24 and the sample solution 402 are simultaneously in the same field of view enabling a very direct visual comparison. In this way when the vial 18 is held up to the light or a white background, the light being transmitted and reflected through both the color standard 24 and the sample solution is exactly the same.

To assist in properly viewing the sample/reactant solution 402 in the vial 18 and the color standard 24, the solution volume in the vial 18 is determined not only to effect an accurate interaction with any lead in the sample 23 so that the effects of dilution are optimized, but also such that the liquid level for the sample solution 402 including the sample 23, first solution 250 and second solution 300 in the vial 18 is located just below the bottom of the color standard 24.

With this combination of sodium hydroxide, optionally triethanolamine, and ammonium sulfide, a graduated range of colors develops in the solution over the range of lead concentrations from 0.1 mg Pb/cm² to 3.0 mg Pb/cm². By testing hundreds of samples with known lead contents, the scale for the color standard 24 was identified and the exact shade of the color standard 24 for various lead concentrations, such as 0.6 mg Pb/cm² and 1.0 mg Pb/cm² was determined. The color of each lead concentration was analyzed digitally to determine the exact ratio of the various pigments required to match these colors so that the color standard 24 could be printed to represent the true color of the solution or solutions with these lead concentrations. Transparent inks are preferred for the printing of the standard 24, which can be printed directly onto the vial or printed on transparent labels for application of the label directly to the bottle. Additionally, while it is preferred to print the color standard 24 on each vial 18 directly above a viewing window, the color standard 24 can also be printed or otherwise represented on a separate card or other structure (not shown) that is to be placed immediately adjacent to the vial 18 for comparison with the sample solution in the vial 18. Alternatively, the sample vial 18 can be placed within a standard colorimeter to measure the wavelengths of light transmitted through the vial 18 to determine the color of the solution in the vial 18 for comparison. By working with the same set of samples with known lead contents the wavelength of light to be used in the colorimeter can be determined along with the % transmittance that corresponds to a given lead concentration. Further, the kit 10 may contain vials 18 printed with different standards 24 corresponding to different lead concentrations to provide indications of various level of lead in the sample 23.

If upon comparing the color of the solution 402 to the test standard 24 immediately after shaking the sample following the sulfide addition the resulting solution 402 is:
Darker than Test Standard: The sample 23 contains more than the specified test kit lead detection level defined by the test standard—test is positive for lead.

If, when initially observed, the resulting solution 402 in the vial 18 is colorless or lighter than the test standard 24, then the vial 18 is set aside for 10 minutes. Then the vial 18 is shaken again and the solution 402 once more compared to the test standard 24 for the final result. If the resulting solution 402 is:
Colorless: The sample 23 contains less than the detection limit of the test, which in the illustrated embodiment is 0.18 g/cm² or 0.0002% w/w
Lighter than Test Standard: The sample 23 contains lead or mercury at less than the specified test kit lead detection level defined by the test standard.
Darker than Test Standard: The sample 23 contains more than the specified test kit lead or mercury detection level defined by the test standard—test is positive for lead or mercury.

After about one half (½) hour a colored solution 402 will begin to clear and after about one (1) to four (4) hours, the solution will revert to colorless again. However the test can be re-run on the solution 402 by adding another five (5) to six (6) drops of the second solution 300 to re-establish the original results. The initial color can be regenerated in a saved sample vial numerous times for at least a year following the initial test.

To verify any negative test result, the kit 10 can also optionally include a number of verification test strips 200 used to verify the strength of the sulfide solution is sufficient for an accurate test. In one embodiment, these test strips 200 are formed from blotting paper or a similarly suitable substrate which is saturated/impregnated with a saturated solution of iron sulfate and dried and cut to form the strips 200. The iron sulfate can react with the solution 402 formed by the sample 23, the first solution 250 and the second solution 300 to provide an indication of the absence of lead in the solution 402. In particular, to verify the negative test result, one drop of the solution 402 of the sample 23, the first solution 250 and the second solution 300 is placed on a strip 200. If the test is negative for lead, the strip 200 will turn black as a result of the interaction of the iron sulfate in the strip with the sulfide ion in solution 402.

The iron sulfate undergoes a simple exchange reaction with the sulfide ion forming the water insoluble iron sulfide compound which is black:

FeSO₄+(NH₄)₂S→FeS(ppt)+(NH₄)₂SO₄

The test strip could be made with any of the cations listed in Table 1, or with lead. A test strip impregnated with lead acetate is too sensitive for this verification as it will detect sulfide concentrations as low as 1 ppm, and we desire a visible reaction that occurs in the vicinity of 500 ppm sulfide. In this embodiment we utilize the iron sulfate due to its low toxicity and its ability to differentiate between sulfide ion concentrations above and below the critical threshold for this test at 450 to 500 ppm.

Alternatively, if the strip 200 is dipped directly into the combined solution in vial 18 of the first solution 250 the second solution 300 and the sample 23, a visible color change can also be observed on the test strip 200 to validate the test results.

Because the resulting solution in the vial 18 containing the sample 23 may be hazardous, e.g., it contains lead, each kit 10 is provided with a waste disposal bag 26 sized to the total contents of each kit 10. Once the test is completed, and the results determined, the used supplies are deposited into the waste bag 26, preferably in the following manner:
1. Used pre-cleaning wipe, as it is potentially contaminated with lead paint dust;
2. Paper 22 as it is potentially contaminated with lead paint dust;
3. Test vial 18 is opened and the test solution 402 poured into the waste disposal bag 26;
4. Test vial 18 along with cap 19;
5. Disposable gloves (not shown) can be added, as they are potentially contaminated; and
6. If any sulfide solution remains in bottle 16 after all tests have been conducted, the bottle 16 is opened, and the surplus sulfide solution is poured in along with the open bottle 16.
   For a test kit 10 with twenty-four (24) test vials 18 including predetermined amounts of the caustic solution and one bottle 16 of the sulfide solution, a one (1) gallon sealable plastic bag 26 is of adequate capacity. This bag 26 will contains a mixture of absorbent material 29 capable of absorbing the entire volume of all of the liquid/solutions contained within the kit 10. The absorbent material 29 can take several different forms, such as kitty litter, a mixture of clay such as bentonite and/or zeolite with a trace of oil (to reduce dusting), shredded paper and/or bedding material, such as processed corn cobs, or combinations thereof. An additional refinement includes the addition of the molar equivalent of an acid to neutralize the total amount of caustics present. While any acid can be used, a powdered acid is preferred, such as monocalcium phosphate, as the dry acid will not reduce the liquid absorbent capacity of the absorbent material(s). Another reason for the preference of the monocalcium phosphate is it can provide phosphate anions to form lead phosphates, which are insoluble in neutral or acidic aqueous solution. In whatever form, the absorbent material 29 functions to absorb the liquid/solutions so that it is no longer a liquid waste, but a solid. In addition, the caustic is neutralized by the acid, when utilized. Further, any free lead or chromate is bound by the clay or zeolite by displacing sodium ions. Any residual sulfide will react with residual lead to form insoluble lead sulfide, or it can react with the iron or aluminum in the clay or zeolite to form insoluble sulfides.

The testing parameters of the kit 10 can be additionally controlled or adjusted according to the desired level of the positive/negative criteria being used for the lead content to be determined, and/or the amount of the various solutions being used for performing the test utilizing the kit 10. The color scale with samples dissolved in the caustic solution and reacted with sulfide ion varies gradually and predictably from completely colorless at 0.0 mg Pb/cm² through linearly, and progressively darkening shades of brown to completely black (with respect to visible light transmittance) at about 3.0 mg Pb/cm², and above this level the shade of black continues to darken with increasing lead and or mercury content to at least 10 mg/cm². The detection parameters of the kit 10 can be varied within this range, or above or below this range by manipulation of one or more of certain attributes of the kit 10.

We have not derived a formula to describe the relationship between all these variables, but as shown below, the threshold for a test result at a particular concentration can be determined by selecting an appropriate value for each of the variables listed below.

For example:
1. Sample size: The amount of sample 23 is controlled by the diameter of the sampling device 20. The paint sample 23 lifts as a plug and provides an equal area around the circumference of each of the paint layers. Varying the sample diameter can be used to adjust the area of the paint or other coating material exposed to the leaching solution to raise or lower the targeted detection limit accordingly.
2. Testing Vial Diameter: The diameter of the test vial 18 can be varied above or below the one inch diameter vial to increase or decrease the color intensity of the sample solution held in the vial 18. For example, for the color standard determined for the detection threshold of 1.0 mg/cm², if the vial diameter is decreased from 1″ to 0.75″ with a corresponding increase in the vial height and the solution volume remains the same so that the volume of the liquid in the vial 18 remains constant, then the pass/fail level for the test is shifted from 1.0 mg/cm² to 1.3 mg/cm². Conversely, if the vial diameter is increased from 1″ to 1.25″ with the liquid volume and color standard fixed, then the pass/fail level for the test is reduced from 1.0 mg/cm² to 0.7 mg/cm² for the color standard selected for a 1″ diameter vial and 1.0 mg/cm² detection color standard.
   3. Color Standard: By comparison of the transmitted color of hundreds of standard paint samples of known lead content with either lead carbonate or lead chromate or both combined, with this test in the standard test vial the color scale was defined. The comparison was done by taking digital photos of the resulting solution color in the vial 18 using transmitted light. In the field of each picture was an 18% grey card so that the exposure of each picture could be verified and then color corrected on the computer. The color corrected photos were then color analyzed to determine the exact level of red, green and blue present. From this data analysis it is possible to select the precise ink color needed to set a different pass/fail color for the color standard 24 that corresponds to the desired color of the lead concentration in the solution in the vial 18. In this manner, with other variables remaining fixed, the pass/fail threshold for the kit 10, or for individual vials 18 in the kit 10 if the kit 10 includes vials 18 for use in the detection of various levels of lead concentrations, can be adjusted up or down as desired.
4. Light Filtering: The relationship between the solution color and the color standard can also be adjusted with the use of a light filter (not shown) to selectively alter the color spectrum of the transmitted light. This light filter by way of example could be composed of acetate, glass or other plastic, for example, and colored red, green, blue, yellow or some other color and placed behind the vial 18 to filter the light before it is transmitted through the solution, so that the light passing through the solution is modified to affect the visual determination of the color comparison with the color standard. Alternately the filter can be placed between the viewer and the solution, or before or after both the vial and color standard. In yet another adaptation, a transparent light filter can be printed on the bottle with transparent ink. The use of the light filter provides an inexpensive means to provide a single standardized test kit with multiple detection limits by altering the color of the solution or the color standard or both. A similar color adjustment can also be effected by providing a colored light source or a colored viewing background.
5. Concentration: The concentration of the alkali metal hydroxide and the alkanolamine can also be varied along with the concentration and amount of sulfide ion added to moderately shift the resulting solution color to make fine tuning adjustments to the color of the resulting solution.

A graphical interpretation of the affects of changes in the variable of the test kit 10 is shown in Table 2:

TABLE 2
Affects of Test Parameter Changes On Detection Limit of Test Kit

| | | Resulting | |
|---|---|---|---|
| | | Change In Lower | |
| | | Detection Limit | Resulting Change |
| | Change in | (Lead/Mercury | In Applicable |
| Parameter | Parameter | Not Present) | Range of Test |
| Size of Sample 23 | Increase | Lowers | Lowers Detection |
| | | | Limit |
| Diameter of Vial 18 | Increase | Raises | Raises Upper Limit |
| Volume of | Increase | Raises | Raises Upper Limit |
| Solution 250 | | | |
| Height of Vial 18 | Increase | Raises | Raises Upper Limit |
| and Volume of | | | |
| Solution 250 | | | |
| Color of Test | Darkened | N/A | Raises Upper Limit |
| Standard 24 | | | |

In terms of the effectiveness of the kit 10 in determining the presence of lead in paint on various household surfaces, the kit 10 has been independently evaluated by the U.S. Environmental Protection Agency, as discussed in the Environmental Technology Verification (ETV) Program Environmental and Sustainable Technology Evaluations Report on the ESCA Tech, Inc. D-LEAD® Paint Test Kit, December 2010, and ETV Verification Statement (available at http://www.epa.gov/nrmrl/std/etv/este.html#pcqstklp), each of which is expressly incorporated by reference herein in its entirety. In brief, the test kit 10 provides accurate results for the detection of lead that far exceed those of prior art tests.

### Verification Test Description

According to the Verification Statement, the verification test of the kit 10 was conducted January through June 2010 at the Battelle Laboratories in Columbus, Ohio. This timeframe included testing of the test kit and also completion of all ICPAES and QC analyses.

Qualitative spot test kits for lead in paint were evaluated against a range of lead concentrations in paint on various substrates using performance evaluation materials (PEMs). PEMs were 3-inch by 3-inch square panels of wood (pine and poplar), metal, drywall, or plaster that were prepared by Battelle. Each PEM was coated with either white lead (lead carbonate) or yellow lead (lead chromate) paint. The paint contained lead targeted at 0.3, 0.6, 1.0, 1.4, 2.0, and 6.0 mg/cm². These lead concentrations were chosen with input from a stakeholder technical panel based on criteria provided in EPA's Lead Renovation, Repair, and Painting (RRP) rule and to represent potential lead levels in homes. Paint containing no lead (0.0 mg/cm²) was also applied to each substrate and tested.

Two different layers of paint were applied over the leaded paint. One was a primer designed for adhesion to linseed oil-based paint and the second coat was a typical interior modern latex paint tinted to one of three colors: white, red-orange, or grey-black. These colors were chosen by EPA, with input from a stakeholder technical panel based on the potential of certain colors to interfere with lead paint test kit operations. The topcoat paint manufacturers' recommended application thickness was used. Two coats at the recommended thickness were applied.

The kit 10 for lead paint was operated by a technical and non-technical operator. The technical operator was a Battelle staff member with laboratory experience who had been trained by the vendor to operate the test kit. The same technical operator operated this test kit throughout testing. Because this lead paint test kit is anticipated to be used by certified remodelers, renovators, and painters, it was also evaluated by a non-technical operator. The non-technical operator was a certified renovator with little to no experience with lead analysis. The non-technical operator was provided the instruction manual, demonstrational DVD, and other materials typically provided by the vendor with the test kit for training. He then viewed the materials himself to understand how to operate the test kit. He was also permitted to ask questions or clarifications of the vendor on the operation of the test kit. This scenario approximated the training that renovators are expected to receive under the RRP rule.

Tests were performed in duplicate on each PEM by each operator, technical and non-technical. Duplicates were tested in succession by each operator on a given PEM. PEMs were analyzed blindly. Test kit operators were not made aware of the paint type, lead level, or substrate of the PEM being tested. PEMs used for analysis were marked with a non-identifying number. PEMs were not tested in any particular order. To determine whether the substrate material affected the performance of the test kits, two unpainted PEMs of each substrate

were tested using each test kit, in the same manner as all other PEMs (i.e., per the test kit instructions). Three PEMs at each lead level, substrate, and topcoat color were prepared for use in this test. Thus, a total of 468 painted PEMs were used in the verification test.

To confirm the lead level of each PEM used for testing, paint chip samples from each PEM were analyzed by a National Lead Laboratory Accreditation Program (NLLAP) recognized laboratory, Schneider Laboratories, Inc., using inductively coupled plasma-atomic emission spectrometry (ICP-AES) as the reference method. The paint chip samples for reference analyses were collected by Battelle according to a Battelle standard operating procedure (SOP), which was based on ASTM E1729. Lead levels determined through the reference analysis were used for reporting and statistical analyses.

The kit 10 was verified by evaluating the following parameters:
1. False positive and negative rates—A false positive response was defined as a positive result when paint with a lead concentration≦0.8 mg/cm² was present. A false negative response was defined as a negative response when paint with a lead concentration≧1.2 mg/cm² was present. Consistent with the EPA's Apr. 22, 2008 RRP rule, panels with lead levels between 0.8 and 1.0 mg/cm² were not used in the false positive analysis, and those with lead levels between 1.0 and 1.2 mg/cm² were not used in the false negative analysis.
2. Precision—Measured by the reproducibility of responses for replicate samples within a group of PEMs. Groups of PEMs evaluated for precision included lead concentrations and substrate material. Responses were considered inconsistent if 25% or more of the replicates differed from the response of the other samples in the same group of PEMs.
3. Sensitivity—The lowest detectable lead level by the test kit 10. This parameter was identified based on the detection results across all PEM levels and was determined based on the lowest PEM lead level with consistent (>75%) positive responses.
4. Modeled Probability of Test Kit Response—Logistic regression models were used to determine the probabilities of positive or negative responses of the test kit at the 95% confidence level, as a function of lead concentration and other covariates, such as substrate type, lead paint type, operator type, and topcoat color. To account for the uncertainty associated with measurement error of the PEMs, the final multivariable model for each test kit was subjected to a simulation and extrapolation (SIMEX) analysis.
5. Matrix Effects—Covariate adjusted logistic regression models were used to determine whether any of the PEMs parameters (topcoat color, substrate, operator, or lead paint type) affected the performance of the test kit. Type III Statistics and comparison of likelihoods from logistic regression models were used to determine the statistical significance of these factors.
6. Operational Factors—Ease of use, operator bias, helpfulness of manuals, technology cost, and sustainability metrics such as volume and type of waste generated from the use of the test kit 10, toxicity of the chemicals used, and energy consumption were noted and summarized. QA oversight of verification testing was provided by Battelle and EPA. Battelle and EPA QA staff conducted technical systems audits and a data quality audit of at least 10% of the test data to ensure that data quality requirements were met. This verification statement, the full report on which it is based, and the test/QA plan for this verification test are available at www.epa.gov/etv/este.html.

### Verification Results

False Positive/Negative Rates: The overall observed false negative rate on PEMs for the kit 10 with confirmed lead levels≧1.2 mg/cm² was 0% for both the technical and non-technical operator. The overall observed false positive rate for the kit 10 on PEMs with confirmed lead levels of ≦0.8 mg/cm² was 16% for the technical operator and 29% for the non-technical operator. The highest individual observed false positive rate came from the non-technical operator testing PEMs with yellow lead paint.

Precision: The kit 10 provided overall consistent responses (either positive or negative) for both the technical and non-technical operator for all lead levels except 0.6 mg/cm². At this level, responses were consistent 54% of the time (i.e., consistently positive 54% of the time). Across all substrates, lead types, and operators, responses produced by the kit 10 on PEMs with confirmed lead levels near 1.0 mg/cm² or greater were consistently positive≧90% of the time. Results on PEMs with lead levels near 0.3 mg/cm² or less were consistently negative 94% of the time or more. Results from the kit 10 indicated 100% precision on PEMs containing no lead and 85% precision on yellow and white lead PEMs.

Sensitivity: The kit 10 was also sensitive down to 1.0 mg/cm² lead for both operator types and all lead levels. This is the lowest sensitivity attainable based on the test design and qualitative nature of the test kits. The kit 10 does, however, provide graded responses to lead concentrations<1.0 mg/cm². The kit 10 has indications for both low lead and no lead responses.

Modeled Probability of Test Kit Response: Based on the lower bound estimates of the modeled probability of the kit 10, the results indicate that, for all possible variable combinations but one, a false negative rate of ≦5% is predicted at 1.2 mg/cm². The highest predicted false negative rate is 5.4% for a technical operator evaluating lead paint with a white topcoat on wood. The modeled probability curve results indicate that at 0.8 mg/cm², there is no combination of variables (operator, substrate, or topcoat) where the upper prediction bound provides a false positive rate of ≦10% for the kit 10.

Matrix Effects: After controlling for the significant covariates, the likelihood of a positive test result is positively and significantly associated with higher lead levels, testing by a non-technical operator, drywall and metal substrates, and a grey topcoat. It is not significantly and positively associated with testing by a technical operator, plaster and wood substrates, or red and white topcoats.

Operational Factors: Both the technical and non-technical operator found the kit 10 instructions to be clear, informative, and easy to follow. The solutions used for different steps were easily identifiable within the kit and the storage conditions of the reagents were readily marked. All reagents came prepared and ready to use.

Over the past three decades many different chemical spot tests for lead in dried coating films have been produced commercially in the US. Previous tests kits have been extensively tested in independent studies e.g. NISTDIR, AIHAJ (supra). These studies all showed that existing test methods were unreliable at showing the presence of lead in dried coating films. In 2008, the US EPA announced (40 CFR §7450) that it would be funding independent third party testing (ETV Testing) of improved chemical spot tests for lead in paint for use by certified renovators. Out of all the chemical spot tests subjected to independent third party tests, only one test, the subject of this patent application and named the D-Lead® Paint Test Kit (i.e., test kit 10) was judged to be reliable at determining when lead was not present in a dried paint film, by reliably showing the presence of lead when it was present, and not indicating lead to be present when it was not.

These test results indicate that the test kit 10:
1. Provides a positive lead test result typically in less than 30 seconds, about the same as the fastest of the other tests.
2. Provides the second fastest method to confirm a negative result that lead is not present at low levels.
3. Works with paints on the widest variety of surfaces, while the sodium sulfide (which does not include a caustic solution 250) test does not work on metal, and the rhodizonate test (which also does not include a caustic solution 250) does not work on drywall or plaster.
4. Works with any paint color.
5. Is the most accurate test. The accuracy data from the independent tests is summarized below. In the interest of brevity, only the results from the two best performing tests from the NISTDIR evaluation of 8 test kits are listed. The results from all four (4) of the kits tested by the ETV Program are listed.
6. The color standard 24 enables an estimation in the concentration of the lead in the sample 23, and with the addition of a color scale or multiple standards 24 reflecting different lead concentrations in the kit 10, the results obtained by the kit 10 can be used to approximate the concentration of lead in the sample 23.
7. The results of the test kit 10 are able to be viewed by even color blind individuals, as a result of the colors of the various solutions 250, 300 and standards 24 employed in the test kit 10.

The results of these tests are summarized in the following Tables 3 and 4 taken from the publication of the results of these NIST/EPA tests:

TABLE 3
Comparison of Independent Test Results of EPA Recognized
Lead Paint Test Kits
False Negative Test Result Percentages

| | | No of false | |
|---|---|---|---|
| Test Kit | # of Tests | Negatives | % |
| D-Lead Paint Test Kit | 1,077 | 8 | 0.7% |
| (test kit 10) | | | |
| (Ref Table 6-3) | | | |
| Sodium Sulfide Test | 186 | 5 | 2.7% |
| Kits 8a & 8b | | | |
| (Ref Table 10B) | | | |
| Rhodizonate Test | 510 | 62 | 12.2% |
| Kits 4a, 4b & 4c | | | |
| (Ref Table 10B) | | | |

in which Ref Table 6-3 data for test kit 10 is taken from Table 6-3, page 24, Environmental Technology Verification (ETV) Program Environmental and Sustainable Technology Evaluations Report on the ESCA Tech, Inc. D-LEAD® Paint Test Kit, December 2010, cited previously and incorporated by reference herein, and Ref. Table 10B data for the sodium sulfide test and the rhodizonate test is taken from Table 10 B, page 30, National Institute for Standards and Technology and the U.S. Department of Housing and Urban Development Office of Lead Hazard Control as reported in: NISTIR 6398, “Spot Test Kits For Detecting Lead in Household Paint: A Laboratory Evaluation,” NIST, May, 2000, also cited previously and incorporated by reference herein.

Additionally, there were a total of four (4) test kits independently tested by the US EPA's Environmental Technology Verification (ETV) Program, including the test kit 10. The results are summarized below in Table 4, and all the results are from Table 6-3 from the respective reports which can be viewed at: http://www.epa.gov/nrmrl/std/etv/este.html#pcqstklp. The ETV testing Program was the most extensive third party independent testing done to date and only the D-Lead Paint Test Kit (i.e., test kit 10) had a statistically accurate ability to detect whenever lead was not present according to the USEPA.

TABLE 4
Comparison of ETV Lead Paint Test Kit Test Results

| | | # of false | |
|---|---|---|---|
| Test Kit | # of Tests | Negatives | % |
| D-Lead Paint Test Kit | 1,077 | 8 | 0.7% |
| (test kit 10) | | | |
| LeadPaintCheck | 1,092 | 27 | 2.5% |
| ANDalyze Lead-in-Paint | 1,072 | 177 | 16.5% |
| Test Kit | | | |
| Lead AVERT Test Kit | 1,064 | 503 | 47.3% |

Additional information concerning the efficacy of the test kit 10 and method of the use of the kit 10 to detect the presence of lead, mercury and chromate is found in the instructional manual developed for use with the test kit 10, a copy of which is available at https://www.esca-tech.com/lptk/pdf/D-Lead Paint Test Manual EN.pdf or https://www.esca-tech.com/lptk/Trainers.php, the contents of which are each expressly incorporated herein by reference in their entirety.

Variations of the above-described embodiments of the present invention are contemplated as being within the scope of the following claims particularly pointing out and distinctly claiming the subject matter regarded as the invention.

We claim:

1. A test kit for determining the presence of lead or mercury in a surface coating material, the kit comprising:
a. at least one container including a caustic solution therein for leaching lead or mercury out of a sample of the surface coating material; and
b. at least one container including a sulfide-ion producing solution therein for reacting with any lead or mercury present in the sample to produce a visible lead or mercury sulfide compound.

2. The test kit of claim 1 further comprising at least one color standard that provides a colorimetric reference for a threshold concentration of lead in the sample.

3. The test kit of claim 2 wherein the color standard is applied directly to the caustic test solution container.

4. The test kit of claim 3 comprising:
a. a plurality of containers including a caustic solution therein for leaching lead or mercury out of a sample of the surface coating material; and
b. a plurality of color standards, one color standard applied to each container.

5. The test kit of claim 4 wherein the color standard on each container represents a different threshold concentration for lead in the sample placed in the container.

6. The test kit of claim 1 further comprising a sample test vial capable of receiving the surface coating sample, the caustic solution and the sulfide-ion producing solution.

7. The test kit of claim 1 wherein the caustic solution includes between about 0.15% w/w and about 15.0% w/w of an inorganic caustic.

8. The test kit of claim 7 wherein the inorganic caustic is an alkali metal hydroxide.

9. The test kit of claim 7 wherein the caustic solution further comprises between about 0.15% w/w and about 15.0% w/w of an alkanolamine.

10. The test kit of claim 1 wherein the sulfide-ion producing solution includes a sulfide compound capable of providing between about 0.03% w/w and 5.0% w/w of a sulfide ion in the sulfide ion-producing solution.

11. A method of determining the presence lead or mercury in a sample of a surface coating, the method comprising the steps of:
a. placing the sample of the surface coating in a container with an amount of a caustic solution;
b. adding an amount of a sulfide-ion producing solution to the container to produce a color in the solution; and
c. comparing the color of a resulting solution in the container with a color standard representative of a solution color for a known concentration of lead in the solution.

12. The method of claim 11 further comprising the step of obtaining a sample of reproducible size from the surface coating prior to placing the sample in the container.

13. The method of claim 12 wherein the step of obtaining the sample comprises driving a sample retrieval device into the surface coating, wherein the device has a tapered end positioned against the surface coating to assist in cutting into the surface coating.

14. The method of the claim 11 further comprising the step of obtaining the sample from the surface covering prior to placing the sample in the container with the amount of a caustic solution.

15. A method for determining the presence of chromate in a sample of a surface coating, the method comprising the steps of:
a. obtaining the sample of the surface coating;
b. placing the sample in a colorless alkaline solution; and
c. determining if a color change has occurred in the solution.

16. A method for determining the presence of lead or mercury in a sample of a surface coating, the method comprising the steps of:
a. obtaining the sample of the surface coating;
b. providing a clear, transparent container including a color standard applied to the exterior of the container;
c. placing the sample and a reagent solution in the container; and
d. determining if a color change has occurred in the solution by visually comparing the color of the solution in the container by light transmission and/or light reflectance with the color standard.
